(19) United States
(12) Patent Application Publication
(10) Pub. No.: US 2005/0053636 A1
(43) Pub. Date: Mar. 10, 2005

(54) Cosmetic and dermatological care oils containing waxes

(75) Inventors: Stephanie Von Der Fecht, Wedel (DE); Jorg Kuther, Pinneberg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/895,972

(22) Filed: Jul. 22, 2004

Related U.S. Application Data
(63) Continuation of application No. PCT/EP03/00537, filed on Jan. 21, 2003.

(30) Foreign Application Priority Data
Jan. 23, 2002 (DE) 102 02 312.3

(51) Int. Cl. A61K 7/025; A61K 7/06

(52) U.S. Cl. 424/401; 424/74

(57) ABSTRACT

A cosmetic or dermatological care oil comprising from 90% to 99.5% by weight of a mineral oil and/or an ester oil and from 0.5% to 10% by weight of a wax.

### CROSS-REFERENCE TO RELATED APPLICATIONS

The present application is a continuation of International Application No. PCT/EH03/00537, filed Jan. 21, 2003, the entire disclosure whereof is expressly incorporated by reference herein, which claims priority under 35 U.S.C. § 119 of German Patent Application No. 102 02 312.3, filed Jan. 23, 2002.

### BACKGROUND OF THE INVENTION

1. Field of the Invention

The present invention relates to cosmetic and dermatological care oils which contain waxes.

2. Discussion of Background Information

The skin is the largest human organ. Amongst its many functions (for example for temperature regulation and as a sensory organ), the barrier function, which prevents the skin (and thus ultimately the entire organism) from drying out, is probably the most important. At the same time, the skin acts as a protective device against the penetration and absorption of external substances. This barrier function is effected by the epidermis which, being the outermost layer, forms the actual protective sheath against the environment. Being about one tenth of the total thickness, it is also the thinnest layer of the skin.

The epidermis is a stratified tissue in which the outer layer, the horny layer (Stratum corneum), is the part which is of significance for the barrier function. The Elias skin model, which is currently recognized in the specialist field (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) correspond to the bricks, and the lipid membrane, which is of complex composition, in the intercellular spaces corresponds to the mortar. This system essentially represents a physical barrier to hydrophilic substances but, because of its narrow and multilayered structure, can equally, however, also be traversed by lipophilic substances only with difficulty.

In one aspect, the present invention relates to cosmetic or pharmaceutical preparations with a reduced feeling of stickiness, to processes for their preparation, and to the use of active ingredients for reducing the feeling of stickiness of cosmetic preparations.

Cosmetic skin care is primarily understood as meaning that the natural function of the skin as a barrier to environmental influences (e.g. dirt, chemicals, microorganisms) and to the loss of endogenous substances (e.g. water, natural fats, electrolytes) is strengthened or restored.

Impairment of this function may lead to increased absorption of toxic or allergenic substances or to attack by microorganisms, leading to toxic or allergic skin reactions.

Another objective of skin care is to compensate for the loss by the skin of sebum and water caused by daily washing. This is particularly important if the natural regeneration ability is inadequate. Furthermore, skin care products should protect against environ- mental influences, in particular against sun and wind, and delay skin ageing.

Medicinal compositions generally comprise one or more medicaments in effective concentrations. For the sake of simplicity, for a clear distinction between cosmetic and medicinal use and corresponding products, reference is made to the legal provisions of the Federal Republic of Germany (e.g. Cosmetics directive, Foods and Drugs Act).

Although it is known to reduce this feeling of stickiness or else feeling of greasiness by adding certain substances, for example a number of selected powder raw materials, in particular talc, apart from the fact that this is only rarely entirely successful, such an addition also changes the viscosity of the respective product and reduces its stability.

It is, therefore, desirable to remedy the above disadvantages. In particular, it would be desirable to have available products with reduced stickiness and/or greasiness. This applies equally to the fields of care cosmetics, decorative cosmetics and pharmacological galenics.

In addition, it would be desriable to develop cosmetic bases for cosmetics preparations which are characterized by good skin compatibility.

Conventional preparations are, for example, care oils. Conventional care oils are, however, difficulty to apply due to their relatively low viscosity. Methods described hitherto for the thickening of oil (through sugar derivates or copolymers) lead to products with high stickiness, low long-term stability and inadequate skin care properties.

Products which are thickened by using block copolymers are known. The use of galactomannan as oil thickener is also known (EP 795 322 and EP 795 323).

### SUMMARY OF THE INVENTION

The present invention provides a cosmetic or dermatological skin care oil which comprises from 90% to 99.5% by weight of a mineral oil and/or an ester oil and from 0.5% to 10% by weight of one or more waxes.

In one aspect, the care oil may comprise at least one ester oil. In another aspect, it may comprise at least one mineral oil. For example, the at least one ester oil may comprise at least one triglyceride oil. The triglyceride oil may in turn comprise a triglyceride based on caprylic acid and/or capric acid and/or it may comprise one or more of olive oil, sunflower oil, soybean oil, peanut oil, rapeseed oil, almond oil, palm oil, coconut oil and palm kernel oil.

In another aspect of the care oil of the present invention, the at least one ester oil may comprise an ester of a carboxylic acid having a chain length of from 3 to 30 carbon atoms and an alcohol having a chain length of from 3 to 30 carbon atoms and/or the at least one ester oil may comprise one or more of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate and erucyl erucate.

In yet another aspect of the care oil of the present invention, the mineral oil may comprise a paraffin oil.

In a still further aspect of the care oil of the present invention, the at least one wax may comprise one or more of a triglyceride wax, montan wax, ceresine, a microcrystalline wax, hydroxyoctacosanyl hydroxystearate, beeswax, synthetic beeswax, and a silicone wax.

In yet another aspect, the at least one wax may comprise an ester of a carboxylic acid having from 10 to 50 carbon atoms and glycerol. For example, the at least one wax may comprise a C₁₈₋₃₆-alkanecarboxylic triglyceride.

In a further aspect, the at least one wax may comprise one or more of glyceryl hydroxystearate, a hydrogenated cocoglyceride, a caprylic/capric/isostearic/adipic triglyceride, glyceryl tribehenate, glyceryl tri(12-hydroxystearate) and hydrogenated castor oil.

In a still further aspect, the at least one wax may comprise a silicone wax, for example, one or more of stearyl dimethicone, cetyl dimethicone, stearoxy dimethicone, behenoxy stearyl dimethicone and stearyl methicone.

In another aspect of the care oil of the present invention, the at least one wax may comprise a natural or synthetic substance having at least all but one of the following properties: kneadable at 20° C., solid to brittly hard, coarsely to finely crystalline, translucent to opaque, but not glass-like, melting above 40° C. without decomposition, of relatively low viscosity and non-stringing even when only a little above the melting point, strongly temperature-dependent consistency and solubility, and polishable under slight pressure.

In another aspect, the care oil of the present invention may further comprise at least one fatty acid salt of one or more of Al, Mg and Zn and/or at least one fatty alcohol and/or at least one antioxidant, for example, tocopherol and/or a derivative thereof, and/or one or more oil-soluble UV filter substances such as, e.g., a 3-benzylidenecamphor derivative, a 4-aminobenzoic acid derivative, 2,4,6-trianilino-(p-carbo-2′-ethyl-1′-hexyloxy)-1,3,5-triazine; an ester of benzalmalonic acid, an ester of cinnamic acid, a benzophenone derivative, a salicylic acid derivative, 1-(4′-tert-butylphenyl)-3-(4′-methoxyphenyl)propane-1,3-dione and 1-phenyl-3-(4′-isopropylphenyl)propane-1,3-dione.

The present invention also provides a cosmetic or dermatological care oil which comprises from 90% to 99.5% by weight of a mineral oil and/or a triglyceride oil and from 0.5% to 10% by weight of one or more of a triglyceride wax, montan wax, ceresine, a microcrystalline wax, hydroxyoctacosanyl hydroxystearate, beeswax, synthetic beeswax, and a silicone wax.

In one aspect, the care oil may comprise olive oil, sunflower oil, soybean oil, peanut oil, rapeseed oil, almond oil, palm oil, coconut oil and/or palm kernel oil.

The present invention also provides a cosmetic base which comprises from 90% to 99.5% by weight of a mineral oil and/or an ester oil and from 0.5% to 10% by weight of one or more wax materials.

Surprisingly, it has been found that cosmetic and dermatological care oils comprising
a) 90-99.5% by weight of one or more oils selected from mineral oils and triglyceride oils,
b) 0.5-10% by weight of one or more waxes overcome the disadvantages of the preparations of the prior art.

In particular, it is surprising that the viscosity of natural and synthetic oils can be increased through waxes and this increase is retained even during prolonged storage and temperature fluctuations. Remarkably, besides excellent applicability, these formulations are characterized by low stickiness, very good skin moisturization and long-lasting care effect.

In accordance with the stipulations of the Deutsche Gesellschaft für Fettwissenschaft [German Society for Fat Science] (Fette, Seifen, Anstrichmittel, 76, 135 (1974)), to designate the term “wax” usually involves taking into consideration the mechanical-physical properties of the waxes that are relevant for their use, while the particular chemical composition is not taken into account for the definition.

“Wax”—like “resin”—is a collective term for a series of natural or synthetically obtained substances which usually have the following properties: kneadable at 20° C., solid to briftly hard, coarsely to finely crystalline, translucent to opaque, but not glass-like, melting above 40° C. without decomposition, even a little above the melting point are of relatively low-viscosity and no-stringing, heavily temperature-dependent consistency and solubility and polishable under slight pressure. If, in borderline cases, a substance does not satisfy more than one of the abovementioned properties, then it is not a wax within the meaning of this definition. Waxes differ from similar synthetic or natural products (e.g. resins, plastic masses etc.) primarily by virtue of the fact that they convert to the molten, low-viscosity state usually between about 50 and 90° C., in exceptional cases also up to about 200° C., and are virtually free from ash-forming compounds.

Advantageous for the purposes of the present invention are, for example, ester waxes, which represent esters of

1. saturated and/or unsaturated, branched and/or unbranched mono- and/or dicarboxylic acids having from 10 to 50 carbon atoms and

2. glycerol.

It may also be advantageous to choose the wax components from glycerides, in particular from triglycerides. The glycerides and triglycerides listed below are particularly advantageous:

| Glyceride | Trade name | Available from |
|---|---|---|
| C₁₆₋₁₈-Triglyceride | Cremeol HF-52- | Aarhus Olie- |
| | SPC | fabrik |
| Glyceryl hydroxystearate | Naturchem GMHS | Rahn |
| Hydrogenated cocoglycerides | Softisan 100 | Hüls AG |
| Caprylic/capric/isostearic/ | Softisan 649 | Dynamit Nobel |
| adipic triglyceride | | |
| C₁₈₋₃₆-Triglyceride | Syncrowax HGLC | Croda GmbH |
| Glyceryl tribehenate | Syncrowax HRC | Croda GmbH |
| Glyceryl tri(12-hydroxystearate) | Thixcin R | Rheox/NRC |
| Hydrogenated castor oil | Cutina HR | Henkel KGaA |
| C₁₆₋₂₄-Triglyceride | Cremeol HF-62- | Aarhus Olie- |
| | SPC | fabrik |

Another example of an advantageous wax for the purposes of the present invention is montan wax. Montan wax is a wax obtained from fossil plant remains present in brown coal, a solid, brittle, hard, conchoidally fracturing, brown-black coloured substance which contains esters of montanic acid with montanyl alcohol and other alcohols, and further comprises resins (15-30%), mineral substances and other substances.

Refined montan wax is white to yellow in colour. Gentle bleaching of crude montan wax with varying amounts of chromic acid produces, by a new bleaching process developed by F. Mader & Co., pale, sometimes virtually colourless, montan derivatives, whose ratio of wax acid : wax ester differs from that of oxidates known hitherto.

A further example of an advantageous wax for the purposes of the present invention is, for example, ceresine. Ceresine (Cera mineralis alba, hard paraffin, paraffinum solidum) is a white, odourless, mass which consists of high molecular weight paraffin hydrocarbons. Ceresine is insoluble in water, soluble in fat solvents and in fats or fat-like substances at elevated temperature, m.p. 50-80°, density 0.91-0.97. Ceresine is generally used for solidifying ointment bases and creams.

A further example of an advantageous wax for the purposes of the present invention is beeswax (synonyms are: Cera flava (yellowish) and Cera alba (white), or in accordance with CTFA: Beeswax) which consists primarily of myricyl palmitate, cerotinic acid, melissic acid, higher alcohols and hydrocarbons. Recent studies suggest that beeswax consists of a homologous series of primarily hexadecanoate alkyl esters between C₃₅ and C₅₄ (“Lexikon der Hilfsstoffe fucr Pharmazie, Kosmetik und angrenzende Gebiete” [Lexicon of auxiliaries for pharmacy, cosmetics and related fields], H. P. Fiedler, 3rd Edition, 1989, Editio Cantor Aulendorf, and sources and cross-references cited therein under the keyword “Synthetisches Bienenwachs” [Synthetic beeswax]).

Beeswax has been an important cosmetic constituent since time began, in particular for creams and ointment preparations, but also for cosmetic pencils. The use of synthetic beeswax in such products is also known.

A further example of an advantageous wax for the purposes of the present invention is synthetic beeswax. Synthetic beeswax comprises, according to R. B. Hutchison, 45-70% by weight of high molecular weight ax-branched monocarboxylic acids, 15-40% by weight of natural or synthetic mixed glycerides of saturated mono- and/or dicarboxylic acids, e.g. glycerol monostearate monoazelate, and also 5-25% by weight of microcrystalline petroleum wax. Such synthetic beeswax is available according to the processes of DE-A 22 41 261 and U.S. Pat. No. 3 914 131.

Another advantageous wax for the purposes of the present invention is hydroxyoctacosanyl hydroxystearate.

Silicone waxes are also advantageous for use as a wax for the purposes of the present invention. It may be advantageous to choose the oil phase of the preparations according to the invention in part or in total from the group of cyclic and/or linear silicones, which are referred for the purposes of the present disclosure as silicone waxes. These are, in particular, silicone waxes, such as, for example, polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Goldschmidt. Also advantageous are the wax grades Wacker-Belsil® SM 6018 (stearyl methicone) and the wax Wacker-Belsil® SDM 6022, which are supplied by Wacker.

As advantages of silicone waxes in the form described here, their consistency, their high chemical resistance and their very good spreadability are particularly notewor- thy.

Advantageous oil components for the purposes of the present invention may be selected, for example, from
mineral oils, mineral waxes
oils, such as triglycerides of capric or caprylic acid, but preferably castor oil;
fats, waxes and other natural and synthetic fatty bodies, preferably esters of fatty acids with alcohols of low carbon number, e.g. with isopropanol, propylene glycol or glycerol, or esters of fatty alcohols with alkanoic acids with low carbon number or with fatty acids;
alkyl benzoates.

The oil components for use according to the present invention may advantageously be selected from esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, from esters of aromatic carboxylic acids and saturated and/or unsaturated, branched and/or unbranched alcohols with a chain length of from 3 to 30 carbon atoms. For example, the ester oils may advantageously be selected from isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g. jojoba oil.

The oil phase may further advantageously be selected from branched and unbranched hydrocarbons and hydrocarbon waxes, silicone oils, dialkyl ethers, saturated or unsaturated, branched or unbranched alcohols, and fatty acid triglycerides, namely the triglyceryl esters of saturated and/or unsaturated, branched and/or unbranched alkanecarboxylic acids with a chain length of from 8 to 24, in particular 12-18, carbon atoms. The fatty acid triglycerides may, for example, advantageously be selected from synthetic, semisynthetic and natural oils, e.g. olive oil, sunflower oil, soybean oil, peanut oil, rape-seed oil, almond oil, palm oil, coconut oil, palm kernel oil and the like.

Any mixtures of such oil and wax components may also advantageous for the purposes of the present invention. It may in some instances also be advantageous to use waxes, for example cetyl palmitate, as the sole lipid component of the oil phase.

Advantageously, the oil phase may also be selected from 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene may particularly advantageously be used for the purposes of the present invention.

It may be advantageous to add auxiliary stabilizers to the care oils according to the invention, particularly advantageously fatty acid salts (advantageously stearates) of Al, Mg and/or Zn, in particular in concentrations of 0.1-2% by weight, and also, fatty alcohols, preferably in concentrations of 0.5-10% by weight.

It may also be advantageous to add antioxidants to the preparations according to the invention. The antioxidants may advantageously be selected from oil-soluble antioxidants, in particular tocopherol and/or its derivatives.

Oil-soluble UV filter substances may also be advantageous, for example: 3-benzylidenecamphor derivatives, preferably 3-(4-methylbenzylidene)camphor, 3-benzylidenecamphor; 4-aminobenzoic acid derivatives, preferably 2-ethylhexyl 4-(dimethylamino)benzoate, amyl 4-(dimethylamino)benzoate; 2,4,6-trianilino-(p-carbo-2′-ethyl-1′-hexyloxy)-1,3,5-triazine; esters of benzalmalonic acid, preferably di(2-ethylhexyl) 4-methoxybenzalmalonate; esters of cinnamic acid, preferably 2-ethylhexyl 4-methoxycinnamate, isopentyl 4-methoxycinnamate; derivatives of benzophenone, preferably 2-hydroxy-4-methoxybenzophenone, 2-hydroxy-4-methoxy-4′-methylbenzophe-none, 2,2′-dihydroxy-4-methoxybenzophenone, and UV filters bonded to polymers, certain salicylic acid derivatives, such as 4-isopropylbenzyl salicylate, 2-ethylhexyl salicylate (=octyl salicylate), homomenthyl salicylate, 1-(4′-tert-butylphenyl)-3-(4′-methoxyphenyl)-propane-1,3-dione and 1-phenyl-3-(4′-isopropylphenyl)propane-1,3-dione.

### DETAILED DESCRIPTION OF THE INVENTION

The examples below are intended to illustrate the present invention.

### EXAMPLE 1

| | % by wt. |
|---|---|
| C₁₈₋₃₆-Alkanecarboxylic | 6.00 |
| triglycerides (Syncrowax ® HGLC) | |
| Beeswax | 1.00 |
| Almond oil | 1.00 |
| Perfume | q.s. |
| Paraffinum liquidum | ad 100.00 |

### EXAMPLE 2

| | % by wt. |
|---|---|
| C₁₈₋₃₆-Alkanecarboxylic | 5.00 |
| triglycerides (Syncrowax ® HGLC) | |
| Beeswax | 0.50 |
| Almond oil | 0.50 |
| Perfume | q.s. |
| Paraffinum liquidum | ad 100.00 |

### EXAMPLE 3

| | % by wt. |
|---|---|
| C₁₈₋₃₆-Alkanecarboxylic triglycerides | 5.00 |
| (Syncrowax ® HGLC) | |
| Dicaprylyl ether | 6.00 |
| C₁₂₋₁₅-Alkyl benzoates | 6.00 |
| Caprylic/capric triglycerides | 6.00 |
| Cyclomethicone | 3.00 |
| 4-Methylbenzylidenecamphor | 1.00 |
| Tocopheryl acetate | 0.50 |
| Perfume | q.s. |
| Butylmethoxydibenzoylmethane | 0.30 |
| Wool wax alcohol | 0.10 |
| Avocado oil | 0.10 |
| Paraffinum liquidum | ad 100.00 |

### EXAMPLE 4

| | % by wt. |
|---|---|
| Octyldodecanol | 6.00 |
| Hydroxyoctacosanyl hydroxystearate | 6.00 |
| Perfume | q.s. |
| Ethylhexyl stearate | ad 100.00 |

### EXAMPLE 5

| | % by wt. |
|---|---|
| C₁₈₋₃₆-Alkanecarboxylic triglycerides | 5.00 |
| (Syncrowax ® HGLC) | |
| Ceresine | 5.00 |
| Zinc stearate | 0.50 |
| Perfume | q.s. |
| Paraffinum liquidum | ad 100.00 |

### EXAMPLE 6

| | % by wt. |
|---|---|
| Stearyl dimethicone and cetyl | 5.00 |
| dimethicone | |
| Stearylmethicones | 0.50 |
| Dimethicones | 40.00 |
| Phenyltrimethicones | 10.00 |
| Almond oil | 1.00 |
| Perfume | q.s. |
| Cyclomethicones | ad 100.00 |

1. A cosmetic or dermatological care oil comprising
(a) from 90% to 99.5% by weight of at least one of a mineral oil and an ester oil, and
(b) from 0.5% to 10% by weight of at least one wax.

2. The care oil of claim 1, wherein the care oil comprises at least one ester oil.

3. The care oil of claim 1, wherein the care oil comprises at least one mineral oil.

4. The care oil of claim 2, wherein the at least one ester oil comprises at least one triglyceride oil.

5. The care oil of claim 4, wherein the at least one triglyceride oil comprises a triglyceride based one at least one of caprylic and capric acids.

6. The care oil of claim 2, wherein the at least one ester oil comprises an ester of a carboxylic acid having a chain length of from 3 to 30 carbon atoms and an alcohol having a chain length of from 3 to 30 carbon atoms.

7. The care oil of claim 2, wherein the at least one ester oil comprises at least one of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate and erucyl erucate.

8. The care oil of claim 4, wherein the at least one triglyceride oil comprises at least one of olive oil, sunflower oil, soybean oil, peanut oil, rapeseed oil, almond oil, palm oil, coconut oil and palm kernel oil.

9. The care oil of claim 4, wherein the mineral oil comprises a paraffin oil.

10. The care oil of claim 1, wherein the at least one wax comprises at least one of a triglyceride wax, montan wax, ceresine, a microcrystalline wax, hydroxyoctacosanyl hydroxystearate, beeswax, synthetic beeswax, and a silicone wax.

11. The care oil of claim 1, wherein the at least one wax comprises an ester of a carboxylic acid having from 10 to 50 carbon atoms and glycerol.

12. The care oil of claim 11, wherein the at least one wax comprises a C₁₈₋₃₆-alkanecarboxylic triglyceride.

13. The care oil of claim 11, wherein the at least one wax comprises at least one of glyceryl hydroxystearate, a hydrogenated cocoglyceride, a caprylic/capric/isostearic/adipic triglyceride, glyceryl tribehenate, glyceryl tri(12-hydroxystearate) and hydrogenated castor oil.

14. The care oil of claim 10, wherein the at least one wax comprises montan wax.

15. The care oil of claim 10, wherein the at least one wax comprises ceresine.

16. The care oil of claim 10, wherein the at least one wax comprises hydroxyoctacosanyl hydroxystearate.

17. The care oil of claim 10, wherein the at least one wax comprises beeswax.

18. The care oil of claim 10, wherein the at least one wax comprises synthetic beeswax.

19. The care oil of claim 10, wherein the at least one wax comprises a silicone wax.

20. The care oil of claim 19, wherein the silicone wax comprises at least one of stearyl dimethicone, cetyl dimethicone, stearoxy dimethicone, behenoxy stearyl dimethicone and stearyl methicone.

21. The care oil of claim 1, wherein the at least one wax comprises a natural or synthetic substance having at least all but one of the following properties: kneadable at 20° C., solid to brittly hard, coarsely to finely crystalline, translucent to opaque, but not glass-like, melting above 40° C. without decomposition, of relatively low viscosity and non-stringing even when only a little above the melting point, strongly temperature-dependent consistency and solubility, and polishable under slight pressure.

22. The care oil of claim 1, wherein the care oil further comprises at least one fatty acid salt of at least one of Al, Mg and Zn.

23. The care oil of claim 1, wherein the care oil further comprises at least one fatty alcohol.

24. The care oil of claim 1, wherein the care oil further comprises at least one antioxidant.

25. The care oil of claim 24, wherein the antioxidant comprises at least one of tocopherol and a derivative thereof.

26. The care oil of claim 1, wherein the care oil further comprises at least one oil-soluble UV filter substance.

27. The care oil of claim 26, wherein the oil-soluble UV filter substance comprises at least one of a 3-benzylidenecamphor derivative, a 4-aminobenzoic acid derivative, 2,4,6-trianilino-(p-carbo-2′-ethyl-1′-hexyloxy)-1,3,5-triazine; an ester of benzalmalonic acid, an ester of cinnamic acid, a benzophenone derivative, a salicylic acid derivative, 1-(4′-tert-butylphenyl)-3-(4′-methoxyphenyl)propane-1,3-dione and 1-phenyl-3-(4′-isopropylphenyl)propane-1,3-dione.

28. A cosmetic or dermatological care oil comprising
(a) from 90% to 99.5% by weight of at least one of a mineral oil and a triglyceride oil,
(b) from 0.5% to 10% by weight of at least one of a triglyceride wax, montan wax, ceresine, a microcrystalline wax, hydroxyoctacosanyl hydroxystearate, beeswax, synthetic beeswax, and a silicone wax.

29. The care oil of claim 28, wherein the care oil comprises at least one of olive oil, sunflower oil, soybean oil, peanut oil, rapeseed oil, almond oil, palm oil, coconut oil and palm kernel oil.

30. A cosmetic base which comprises
(a) from 90% to 99.5% by weight of at least one of a mineral oil and an ester oil,
(b) from 0.5% to 10% by weight of at least one wax.
